(19) United States
(12) Patent Application Publication
(10) Pub. No.: US 2003/0060892 A1
(43) Pub. Date: Mar. 27, 2003

(54) Bone filler material

(76) Inventors: Paul Wilhelm Richter, Pretoria (ZA); Ugo Ripamonti, Sandton (ZA); Michael Edward Thomas, Pretoria (ZA)

Correspondence Address: FROMMER LAWRENCE & HAUG, 745 FIFTH AVENUE- 10TH FL., NEW YORK, NY, 10151, US

(21) Appl. No.: 10/255,737

(22) Filed: Sep. 25, 2002

Related U.S. Application Data
(63) Continuation of application No. PCT/IB02/00182, filed on Jan. 23, 2002.

(30) Foreign Application Priority Data
Feb. 2, 2001 (ZA) 2001/0945

(51) Int. Cl. A61F 2/28

(52) U.S. Cl. 623/23.51; 106/35; 623/23.56

(57) ABSTRACT

A bone filler material includes a plurality of particles of bioactive material. A majority of these particles are individually produced particles.

This invention relates to a bone filler material.

According to the invention, there is provided a bone filler material which includes a plurality of particles of bioactive material, with a majority of these particles being individually produced particles.

By ‘individually produced’ in respect of a particle of the bioactive material is meant that each such particle has been individually manufactured, such as by moulding or extrusion, to have a specific shape and size.

The bone filler material of the invention is used for in vivo filling of defects or gaps in bone in humans or animals. The bone filler material is used as such, or mixed with other components such as blood-derived products which act as a binder, to pack into bone defects such as gaps or cavities. The material is packed or placed surgically, ie by implantation, or by injection, in which case it is used in the form of an injectable formulation.

Preferably, substantially all, ie about 100%, of the particles of bioactive material present in the bone filler material may be individually produced particles. It will, however, be appreciated that the bone filler material may contain no more than a minor proportion of particles of bioactive material which are not individually produced particles, such as particles derived from attrition or break-up of the individually produced particles. Typically, the bone filler material may thus comprise at least 95% by mass individually produced particles of bioactive material, and 5% or less particles of bioactive material derived from attrition or break-up of the individually produced particles.

Each individually produced particle will thus comprise a body of the bioactive material, with the body having said specific shape and size and having an outer surface which is bioactive.

The bodies of the individually produced particles may be of irregular shape or, preferably, of regular shape such as cubic, parallelepiped, cylindrical or spherical shape.

The bodies of the individually produced particles may be sized such that their maximum dimension is at most about 3 mm, typically 1-2 mm. Thus, for example, when the bodies are cubic shaped, the lengths of their individual sides may be about 1.5 mm; when they are cylindrically shaped, the lengths of the cylinders may be about 1 mm and their diameters may be about 1 mm; when they are spherical, the outer diameters of the bodies may be from 0.5-1 mm. However, these dimensions can be varied as desired provided that the material can be used for filling defects or gaps in bone by implantation or can be used in the form of an injectable formulation.

The individually produced particles are thus of a suitable bioactive material, and are osteoconductive. The individually produced particles may even be osteoinductive, such as in a particular arrangement of the particles, eg a cluster or pocket of the particles, tightly bound together. In one embodiment of the invention, the bioactive material may be a non-resorbable material, such as hydroxyapatite. In another embodiment of the invention, the bioactive material may be a partially resorbable material, such as a hydroxyapatite-tricalcium phosphate composite material. In yet another embodiment of the invention, the bioactive material may be a fully resorbable material, such as tricalcium phosphate.

At least one cavity may be provided in the body of each individually produced particle, with the cavity being in communication with the outer surface.

In one embodiment of the invention, the or each cavity in each individually produced particle may be in the form of a surface indentation or concavity. Each surface indentation or concavity may be hemispherical so that it is in the form of a dimple. The radii of the dimples may be from 200-400 μm, or possibly even greater. Preferably, as many dimples as possible are provided on each individually produced particle, subject to the constraints of the manufacturing method used. Thus, for example, when each individually produced particle is cubic shaped and is manufactured by moulding, a dimple can be provided in each side of the body. However, when each individually produced particle is cubic shaped and is manufactured by extrusion, a dimple will typically be provided in only four of the sides.

In another embodiment of the invention, the cavity or cavities may occupy a substantial proportion of the volume of the particle. In other words, the cavity volume, ie the volume of the cavity or the combined volume of the cavities when the particle has more than one cavity, constitutes a substantial proportion of the volume of the particle. Typically, the cavity volume may constitute at least 50% of the volume of the particle. In one version, the body may then be spherical, and may, in particular, be in the form of a hollow spherical shell having an access opening, and with the shell thus being of bioactive material. In another version, the body may be of irregular shape, and a plurality of cavities may be provided within the body. The bone filler material may even comprise a mixture of such spherically and irregularly shaped individually produced particles. The spherically shaped particles may then, in such a mixture, be smaller than the irregularly shaped particles.

The invention will now be described in more detail with reference to the accompanying drawings and non-limiting examples.

In the drawings,

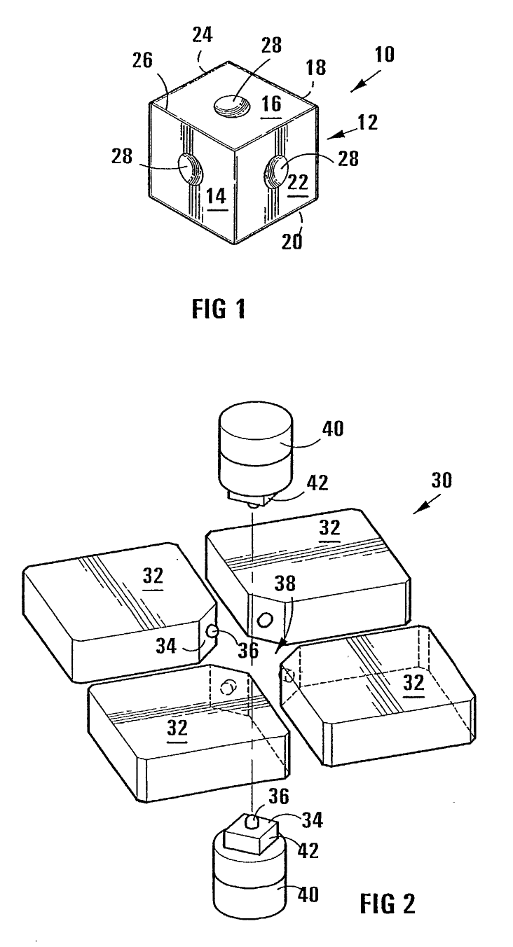

FIG. 1 shows a three-dimensional view of an individually produced particle of a bone filler material according to a first embodiment of the invention;

FIG. 2 shows, in part, a three-dimensional simplified or diagrammatic exploded view of apparatus for producing or manufacturing the particle of FIG. 1;

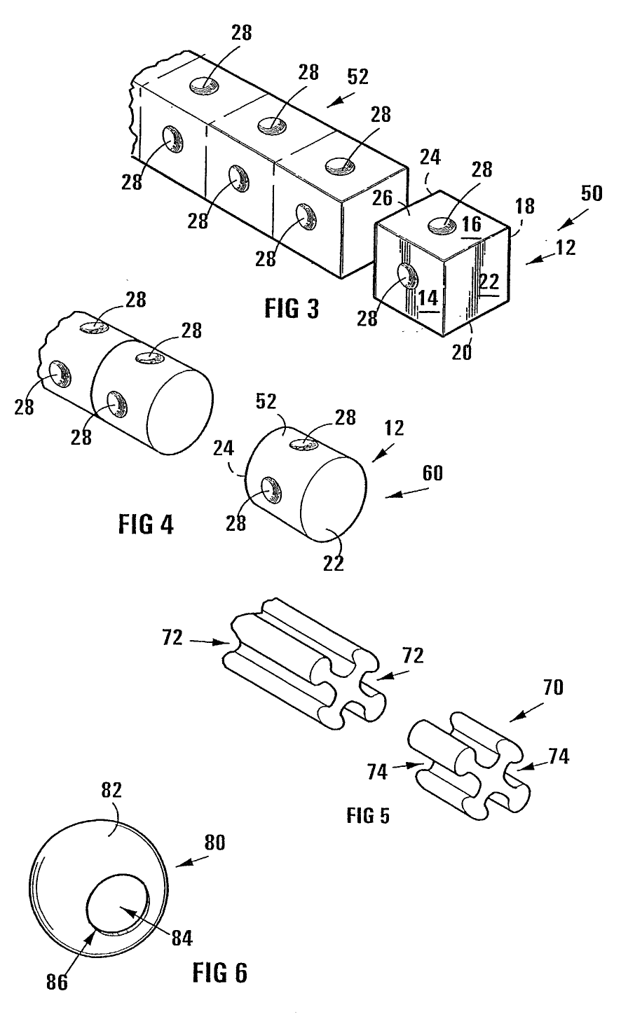

FIG. 3 shows a three-dimensional view of an individually produced particle of a bone filler material according to a second embodiment of the invention;

FIG. 4 shows a three-dimensional view of an individually produced particle of a bone filler material according to a third embodiment of the invention;

FIG. 5 shows a three-dimensional view of an individually produced particle of a bone filler material according to a fourth embodiment of the invention; and

FIG. 6 shows an individually produced particle of a bone filler material according to a fifth embodiment of the invention.

Referring to FIGS. 1 and 2, reference numeral 10 generally indicates an individually produced particle of a bone filler material according to a first embodiment of the invention.

The particle 10 comprises a solid cubic shaped body 12 of hydroxyapatite. The body 12 has surfaces 14, 16, 18, 20, 22 and 24. The edges along which adjacent surfaces meet, such as the edge 26 along which the surfaces 14, 16 meet, are about 1.5 mm long.

In each of the surfaces there is provided a hemispherical dimple 28 having a radius of 200-400 μm.

The particle 10 is produced in a die, generally indicated by reference numeral 30.

The die or mould 30 comprises four solid die pieces 32. Each die piece 32 is square when seen in plan view and has a mould or die face 34 from which protrudes a rounded protrusion 36. The die pieces 32 are arranged such that they define between them a mould cavity 38.

The die 30 also includes a pair of opposed pistons 40. Each piston 40 also has a die face 34 and a protrusion 36.

In use, a hydroxyapatite powder with a mean particle size of about 1 μm is mixed, at an elevated temperature of 120° C., with a thermoplastic binder suitable for extrusion, injection moulding or pressing. The mixture is crushed and sieved to obtain a coarse powder with particles smaller than 300 μm. This powder is hereinafter referred to as the ‘base material’ and was used to produce all the individually produced particles referred to herein with reference to FIGS. 1 to 6.

The die 30 is thus split in the horizontal direction into the four die pieces 32. When the die pieces 32 are assembled and clamped, the mould cavity 38 of square cross section is formed. Each hemispherical protrusion 36 on a die face 34 will cause an indentation or dimple 28 to be formed in the corresponding four sides of a compact that is pressed in the die. Each of the pistons 40 has a square cross section portion 42 which fits slidingly into the mould cavity 38. The portions 42 are provided with the die faces 34 and hemispherical protrusions 36 so that, by means of these hemispherical protrusions 36, hemispherical indentations or dimples will be formed in the top and bottom faces of a compact when pressed in the cavity 38.

Thus, to produce the particle 10, the die 30 is assembled and clamped (not shown), the bottom piston 40 located in position with its portion 42 inserted in the cavity 38, the cavity 38 loaded with the base material, the top piston 40 inserted, and the powder compacted by applying hydraulic pressure to the two pistons. On disassembly of the die, a compact in the shape of a cube is obtained, with the compact having indentations on each of the flat surfaces of the cubic body. The compact is placed in a furnace and fired to remove the thermoplastic binder and to sinter the hydroxyapatite particles. After sintering, the body can be subjected to mechanical abrasion by tumbling or to acid treatment to remove sharp edges and corners. This involves immersion in dilute citric acid for several minutes, rinsing in water and firing to a low temperature of about 500° C. to dry the body and remove the acid residue. The resultant manufactured product is the individually produced particle 10.

A bone filler material according to the invention will thus comprise a plurality of the particles 10, ie a bulk quantity of the particles 10. In other words, it will comprise at least 95% by mass of the particles 10, and 5% or less by mass of particles of bioactive material derived from attrition of the particles 10. The filler material is used for filling defects or gaps in bone. To achieve this, the filler material is used as is, or mixed with other components such as blood derived products which act as a binder, and packed into bone defects such as gaps or cavities. The packing can be effected surgically, ie by implantation, or the filler material can be made up into an injectable formulation which is then located in position by injection.

Referring to FIG. 3, reference numeral 50 generally indicates an individually produced particle of a bone filler material according to a second embodiment of the invention.

Parts of the particle 50 which are the same or similar to those of the particle 10 hereinbefore described, are indicated with the same reference numerals.

The particle 50 is the same as the particle 10 save that it does not have dimples 28 in its surface 22 and its surface 24. This is as a result of its method of production or manufacture.

The particle 50 is produced by extruding the base material through a nozzle (not shown) with a square orifice to produce a continuous strand 52 of green extrudate of square cross section. On exiting the nozzle, the extrudate is indented by four actuatable pins (not shown) spaced 90° apart, and thereafter chopped or cut to a desired length. This results in components of cubic shape with hemispherical dimples 28 on the four sides 14, 16, 18 and 20. These components can be fired and subjected to mechanical abrasion or to acid treatment as described hereinbefore with reference to FIGS. 1 and 2, to remove sharp edges and corners. In this fashion the individually produced particles 50 are manufactured. It will be appreciated that, while the particles 50 will be generally cubic shaped, due to the fact that they are manufactured by extrusion, their bodies may be distorted to a degree so that they are not perfectly symmetrical.

Furthermore, for practical considerations, the actuatable pins will not normally be located at the same level so that the four indentations or dimples 28 will not usually be located equidistantly from the surfaces 22, 24. Furthermore, one or more of the surfaces 14, 16, 18 and 20 may contain only a portion of a dimple or portions of two dimples, depending on where the cutting of the green extrudate is effected.

Referring to FIG. 4, reference numeral 60 generally indicates an individually produced particle of a bone filler material according to a third embodiment of the invention.

Parts of the particle 60 which are the same or similar to those of the particles 10, 50 hereinbefore described, are indicated with the same reference numerals.

The particle 60 is substantially the same as the particle 50, and is produced in the same manner, ie by extrusion; however, in this case a circular extrusion nozzle is used so that the body 12 of the particle 60 is circular in cross section so that it has an outer cylindrical surface 52 between its surfaces 22, 24. The particle 60 thus does not have dimples 28 in its surfaces 22 and 24 but does have four dimples 28 spaced circumferentially apart on its outer cylindrical surface 52. Instead, a greater or lesser number of dimples (not shown) can be provided.

Referring to FIG. 5, reference numeral 70 generally indicates an individually produced particle of a bone filler material according to a fourth embodiment of the invention.

Parts of the particle 70 which are the same or similar to those of the particles 10, 50, 60 hereinbefore described, are indicated with the same reference numerals.

The particle 70 is formed in similar fashion to the particles 50 and 60, ie by extrusion, save that the extrusion nozzle has an orifice which is in the shape of a Maltese cross having arms whose ends or extremities are rounded rather than forked as indicated in FIG. 5. The base material is extruded through the orifice to form a continuous strand of a green extrudate containing four channels or flutes 72. On exiting the nozzle, the green extrudate is chopped to the desired length. The chopping blade (not shown) deforms the extrudate such that the channels are pinched closed (not shown) at the respective ends of the particle, thereby creating four cavities 74 which are approximately hemispherical in shape. The top components are then fired and subjected to mechanical abrasion or to acid treatment as described hereinbefore with reference to FIGS. 1 and 2, to remove sharp edges and corners.

Referring to FIG. 6, reference numeral 80 generally indicates an individually produced particle of a bone filler material according to a fifth embodiment of the invention.

The particle 80 is spherical, and comprises a hollow shell 82 of hydroxyapatite. A cavity 84, which occupies substantially more than 80% of the volume of the particle 80, is thus provided within the shell 82. The shell 82 has an access opening 86 by means of which the cavity 84 is in communication with the outer surface of the shell 82.

The particle 80 is produced by mixing the base material with more-or-less spherical particles of a fugitive phase material, ie a material that will decompose and volatilize during firing. The fugitive phase material can, for example, be stearic acid balls. The resultant mixture is rolled or stirred so that the base material adheres as a coating to the fugitive phase spheres, and excess base material is removed. The thus coated spheres are placed on an absorbent fibre board such as an alumina fibre board, and fired to remove the fugitive phase. This produces the hollow sintered shell with the large access opening 86 which is produced by the outflow of molten fugitive material during firing of the coated sphere. The substantially spherical outer shape of the particle 80 is believed to be particularly suitable for injectable formulations where good flow and low solid volume of the filler material particles is advantageous.

If desired, a plurality, eg 3 or 4, of the particles 80 can be located adjacent one another in abutting relationship, prior to firing of the particles. On firing, the shells 82 of adjacent particles sinter together while each shell remains more-or-less intact, thus retaining its cavity 84 and access opening 86. In this fashion, an irregularly shaped individually produced particle, having a plurality of the cavities 84, is obtained. Such an irregularly shaped individually produced article can thus also be considered to be a cluster or pocket of a plurality of the particles 10, tightly bound together. The bone filler material of the invention can then comprise a mixture of the spherical particles 80 and the irregularly shaped particles, with the spherical particles 80 being smaller than the irregularly shaped particles. Hitherto, bone filler materials have comprised granules of bioactive or biocompatible material. The granules are obtained by crushing the bioactive or biocompatible material in porous form, and sieving the crushed product to extract fragments within a desired size range In bone filler materials thus obtained, the granule shape is not predictable, the granules have multiple sharp fracture points on their outer surfaces, and much material is wasted.

In contrast, with the individually produced particles according to the invention, the particle shape and size is predictable, there are few (if any) sharp edges, and there is minimal wastage of material.

1. A bone filler material which includes a plurality of particles of bioactive material, with a majority of these particles being individually produced particles.

2. A bone filler material according to claim 1, wherein substantially all of the particles of bioactive material present therein are individually produced particles, with the bone filler material containing at most a minor proportion of particles of bioactive material which are not individually produced particles.

3. A bone filler material according to claim 1, wherein each individually produced particle comprises a body of the bioactive material, with the body having a regular shape and an outer surface which is bioactive.

4. A bone filler material according to claim 1, wherein each individually produced particle comprises a body of the bioactive material, with the body having an irregular shape and an outer surface which is bioactive.

5. A bone filler material according to claim 3, wherein the bodies of the individually produced particles are sized such that their maximum dimension is at most 3 mm.

6. A bone filler material according to claim 3, wherein the bioactive material is non-resorbable.

7. A bone filler material according to claim 6, wherein the bioactive material is hydroxyapatite.

8. A bone filler material according to claim 3, wherein the bioactive material is partially resorbable.

9. A bone filler material according to claim 8, wherein the bioactive material is a hydroxyapatite-tricalcium phosphate composite material.

10. A bone filler material according to claim 3, wherein the bioactive material is fully resorbable.

11. A bone filler material according to claim 10, wherein the bioactive material is tricalcium phosphate.

12. A bone filler material according to claim 3, wherein at least one cavity is provided in the body of each individually produced particle, with the cavity being in communication with the outer surface of the body.

13. A bone filler material according to claim 12, wherein the cavity in the body of each individually produced particle is in the form of a surface indentation or concavity

14. A bone filler material according to claim 13, wherein the surface indentation or concavity in the body of each individually produced particle is hemispherical so that it is in the form of a dimple in the surface of the body.

15. A bone filler material according to claim 14, wherein a plurality of the dimples are provided in the body of each individually produced particle.

16. A bone filler material according to claim 12, wherein the cavity volume of each individually produced particle constitutes a substantial proportion of the volume of the particle.

17. A bone filler material according to claim 16, wherein the cavity volume of each individual produced particle constitutes at least 50% of the volume of the particle.

18. A bone filler material according to claim 17, wherein the body of each individually produced particle is spherical, and is in the form of a hollow spherical shell having an access opening, with the shell thus being of the bioactive material.

19. A bone filler material according to claim 17, wherein the body of each individually produced particle is of irregular shape, with a plurality of the cavities being provided within the body.
